(19) United States
(12) Patent Application Publication
(10) Pub. No.: US 2022/0370531 A1
(43) Pub. Date: Nov. 24, 2022

(54) SELF-EMULSIFYING CANNABINOID FORMULATION AND METHOD

(71) Applicant: LANDRACE BIOSCIENCE INC., Chattanooga, TN (US)

(72) Inventors: Honorio Velasco Obias, Ooltewah, TN (US); Eumelia Villegas Tipa, Ooltewah, TN (US); John Christopher Demoss, Rising Fawn, GA (US); John Peter Hetzler, Chattanooga, TN (US); Ryan James Piersant, Signal Mountain, TN (US)

(21) Appl. No.: 17/774,089

(22) PCT Filed: Nov. 3, 2020
(86) PCT No.: PCT/US2020/058733

§ 371 Date: May 3, 2022

Related U.S. Application Data
(60) Provisional application No. 62/930,401, filed on Nov. 4, 2019.

(51) Int. Cl. A61K 36/185; A61K 9/107; A61K 47/10; A61K 47/22; A61K 47/40; A61K 47/24; A61K 31/05

(52) U.S. Cl. CPC A61K 36/185; A61K 9/1075; A61K 47/10; A61K 47/22; A61K 47/40; A61K 47/24; A61K 31/05

(57) ABSTRACT

A composition is provided, including a self-emulsifying pharmaceutical formulation comprising from about 0.001% to about 40% by weight of a cannabinoid or a cannabinoid blend, from about 1% to about 10% by weight of a non-ionic surfactant solubilizer, from about 0.1% to about 10% by weight of an ionic co-surfactant solubilizer, from about 0.001% to about 80% by weight of a first partitioning solubilizer, from about 0.001% to about 70% by weight of a second partitioning solubilizer, from about 0.001% to about 30% by weight of a clathrate solubilizer, from about 0.01% to about 80% by weight of a clarifying solubilizer, and from about 0.001% to about 20% by weight of a sensate.

### CROSS-REFERENCE

This application claims the benefit of U.S. Provisional Application No. 62/930,401, filed Nov. 4, 2019, the contents of which is incorporated by reference herein in its entirety.

### BACKGROUND

A composition is described for administering cannabinoids, cannabinoid extract, or other standardized marijuana extracts to patients and, more particularly, a composition including self-emulsifying pharmaceutical formulations which optimizes cannabinoid or cannabinoid extract dissolution properties thereby enhancing bioavailability of cannabinoid and other standardized marijuana extracts. Methods of making and using the self-emulsifying pharmaceutical formulations are also described.

Cannabinoids are a class of active compounds derived from the Cannabis sativa, Cannabis indica, or Cannabis hybrid plant commonly known as marijuana. The plant contains more than 400 chemicals and approximately 60 cannabinoids. The most notable chemical compound of the naturally occurring cannabinoids is phytocannabinoid tetrahydrocannabinol (THC), particularly Δ⁹-THC, the primary psychoactive compound in cannabis. Cannabidiol (CBD) is another cannabinoid and major constituent of the cannabis plant. Cannabinoids such as THC and CBD are sometimes used for the treatment of various medical conditions. Medical cannabis is used for treating and alleviating symptoms associated with a growing number of indications, including pain, anorexia, asthma, glaucoma, arthritis, spasms, anxiety, and substance withdrawal. Many other illnesses are emerging as potential cannabis-responsive indications.

Cannabinoids are hydrophobic and may have low or variable bioavailability in some formulations. To compensate, a pharmaceutical formulation including cannabionoids may utilize or take advantage of one or more mechanisms to increase the rate or the extent to which the administered cannabinoid is absorbed. To overcome low bioavailability, various lipid-based drug delivery systems, and particularly self-emulsifying drug delivery systems (SEDDS) have been demonstrated to increase the solubility, dissolution and bioavailability of many insoluble drugs. SEDDS are isotropic mixtures of drugs, lipids and surfactants, and may have one or more hydrophilic co-emulsifiers that form fine oil in water emulsions upon mild agitation in an aqueous medium. For example, self-emulsifying products may spontaneously emulsify in vivo. The SEDDS may be self-nanoemulsifying drug delivery system (SNEDDS). Nano-emulsions, defined as having a droplet size of up to 200 nm, may improve bioavailability by increasing the drug solubility, enhancing permeation across the intestinal membrane through a wide distribution in the gastrointestinal tract due to the small droplet size and decreasing food effect on bioavailability. Delivery systems are disclosed in several publications, including WO 2019/135225, WO 2020/037412, WO 2020/118415, and U.S. Patent Application Publication Nos. 2016/0184258, 2019/0298683, 2020/0102131, 2020/0246404, the contents of all of which are incorporated herein in their entirety.

Demand for pharmaceutical formulations including one or more cannabinoids is expected to increase. Consumer acceptance of cannabinoid products will follow product formats that are consumer-friendly, convenient and easy to use, such as liquids, gels, and soft solids. For rexample, product lines available from Landrace Bioscience Inc. of Chattanooga, TN, comprise multiple active pharmaceutical ingredients in self-emulsifying nanodelivery system technologies for prescription healthcare and over-the-counter applications. These technologies cover multiple forms, including liquids, soft solids such as gummies and soft chews, and other chewable forms. Self-emulsifying cannabinoid nanodelivery system technology renders the cannabinoids readily bioavailable in an oral dosage form or topically for fast absorption and targeted delivery to a desired treatment location.

For the foregoing reasons, there is a need for a composition for conveniently administering therapeutically-effective amounts of cannabinoids, cannabinoid extracts or other standardized marijuana extracts. The composition should include pharmaceutical formulations which allow delivery via an oral gastrointestinal dosage form, as well as other forms of delivery such as topical applications. The composition should provide sufficient bioavailability of cannabinoids or standardized marijuana extracts for the treatment of numerous medical conditions for which the active ingredients can be therapeutically beneficial.

### SUMMARY

A composition is provided, including a self-emulsifying pharmaceutical formulation comprising from about 0.001% to about 40% by weight of a cannabinoid or a cannabinoid blend, from about 1% to about 10% by weight of a non-ionic surfactant solubilizer, from about 0.1% to about 10% by weight of an ionic co-surfactant solubilizer, from about 0.001% to about 80% by weight of a first partitioning solubilizer, from about 0.001% to about 70% by weight of a second partitioning solubilizer, from about 0.001% to about 30% by weight of a clathrate solubilizer, from about 0.01% to about 80% by weight of a clarifying solubilizer, and from about 0.001% to about 20% by weight of a sensate.

In one embodiment, the cannabinoid or cannabinoid blend is an amount of from about 0.01% to about 30% by weight. In one aspect, the cannabinoid or cannabinoid blend is an amount of from about 2% to about 20% by weight. In another aspect, the cannabinoid or cannabinoid blend is an amount of from about 2% to about 10% by weight.

In another embodiment, the non-ionic surfactant solubilizer is an amount of from about 0.03% to about 40% by weight. In one aspect, the non-ionic surfactant solubilizer is an amount of from about 8.5% to about 30% by weight. In another aspect, the non-ionic surfactant solubilizer is an amount of from about 9% to about 22% by weight.

In still another embodiment, the ionic surfactant solubilizer is an amount of from about 0.1% to about 15% by weight. In one aspect, the ionic surfactant solubilizer is an amount of from about 0.5% to about 5% by weight.

In yet another embodiment, the partitioning solubilizer is an amount of from about 0.01% to about 80% by weight. In one aspect, the partitioning solubilizer is an amount of from about 25% to about 75% by weight. In another aspect, the partitioning solubilizer is an amount of from about 42% to about 63% by weight.

In a further embodiment, the second partitioning solubilizer is an amount of from about 0.01% to about 70% by weight. In one aspect, the second partitioning solubilizer is an amount of from about 14% to about 50% by weight. In another aspect, the second partitioning solubilizer is an amount of from about 20% to about 29% by weight.

In an additional embodiment, the clathrate solubilizer is an amount of from about 0.01% to about 50% by weight. In one aspect, the clathrate solubilizer is an amount of from about 5% to about 20% by weight.

In another embodiment, the general solubilizer is an amount of from about 0.01% to about 80% by weight In one aspect, the general solubilizer is an amount of from about 10% to about 60% by weight. In another aspect, the general solubilizer is an amount of from about 15% to about 45% by weight.

In still another embodiment, the sensate is an amount of from about 0.01% to about 30% by weight. In one aspect, the sensate is an amount of from about 0.1% to about 15% by weight. In another aspect, the sensate is an amount of from about 0.1% to about 5% by weight.

In one embodiment, the weight ratio of partitioning solubilizer to total surfactant ratio is from about 1.5 to about 10. In one aspect, the weight ratio of partitioning solubilizer to total surfactant ratio is from about 3 to about 9.

In another embodiment, the ratio of weight of cannabinoid to volume of the formulation is greater than about 25.

In one embodiment, the non-ionic surfactant solubilizer is selected from poloxamer 184, poloxamer 334, poloxamer 407, polysorbate 20, polysorbate 60, polysorbate 80, polyoxol 29, polyoxol 35, polyoxol 40, polyoxol 200, and combinations thereof.

In another embodiment, the ionic surfactant solubilizer is selected from soy lecithin, phosphatidyl choline, phospholipids, and combinations thereof.

In still another embodiment, the first partitioning solubilizer comprises dimethyl isosorbide.

In yet another embodiment, the second partitioning solubilizer comprises transcutol HP.

In a further embodiment, the clathrate solubilizer comprises beta-cyclodextrin.

In additional embodiment, the clarifying solubilizer is selected from 1,3-propanediol, propylene glycol, glycerin, triacetin, diacetin, triethylcitrate, and combinations thereof.

In another embodiment, the sensate is selected from natural flavors, artificial flavors, fragrances, terpenes, and combinations thereof.

A method of using a composition including a self-emulsifying pharmaceutical formulation is provided. The method of use comprises the step of administering a therapeutically effective amount of the formulation to a subject for treatment of cannabis-responsive indications.

### DESCRIPTION

The term “cannabinoid” as used herein generally refers to one of a class of diverse chemical compounds that act on a cannabinoid receptor in cells that repress neurotransmitter release in the brain. The term “cannabinoid” as used herein further refers to a class of chemical compounds that has a structure similar to the structure of a compound acting on a cannabinoid receptor in cells. Ligands for these receptor proteins include the endocannabinoids produced naturally in the body by humans and animals, the phytocannabinoids found in cannabis and some other plants, and synthetic cannabinoids manufactured artificially. In certain embodiments, the cannabinoid is a natural cannabinoid. In certain embodiments, the cannabinoid is a natural cannabinoid found in a Cannabis plant. In certain embodiments, the cannabinoid is a synthetic cannabinoid. In certain embodiments, the cannabinoid is a mixture of natural cannabinoids. In certain embodiments, the cannabinoid is a mixture of synthetic cannabinoids. In certain embodiments, the cannabinoid is a mixture of natural and synthetic cannabinoids.

The phrase “cannabinoid-responsive symptom, disease or disorder” as used herein refers to any symptom, disease or disorder which is associated with therapeutic benefit by a cannabinoid, by a mixture of cannabinoids, or by extracts of cannabis plants.

The term “self-emulsifying composition” as used herein refers to a composition that forms an emulsion when placed in an aqueous medium. A self-emulsifying composition is not by itself an emulsion, i.e. it does not comprise a mixture of two or more liquids that are normally immiscible (unmixable or unblendable).

In one embodiment, pharmaceutical compositions and formulations comprising cannabinoids are provided which quickly self-emulsify upon hydration in biological fluids under physiological conditions and produce fine particles for releasing the load of cannabinoids into emulsion form thus enhancing bioavailability. In another embodiment, the composition is a pharmaceutical formulation. In another embodiment, the composition or pharmaceutical formulation is a dosage form, such as a liquid, a solid or a semi-solid dosage form for, for example, oral delivery of cannabinoids. In yet another embodiment, the composition or pharmaceutical formulation is a dosage form, such as a liquid or a semi-solid dosage form for, for example, topical delivery of cannabinoids, such as in a lotion or spray.

The cannabinoids are dispersed in a self-emulsifying medium including at least one surfactant to promote self-emulsification and at least one hydrophilic co-solvent. Referring to Table 1, the self-emulsifying dosage form includes (a) from about 0.001% to about 40% by weight of a pharmaceutically active cannabinoid or a cannabinoid blend; (b) from about 1% to about 10% by weight of a non-ionic surfactant solubilizer which promotes self-emulsification; (c) from about 0.1% to about 10% by weight of an ionic co-surfactant solubilizer; (d) from about 0.001% to about 80% by weight of a first partitioning solubilizer; (e) from about 0.001% to about 70% by weight of a second partitioning solubilizer; (f) from about 0.001% to about 30% by weight of a clathrate solubilizer; (g) from about 0.01% to about 80% by weight of a clarifying solubilizer; and (h) from about 0.001% to about 20% by weight of a sensate, wherein the composition self-emulsifies in an aqueous medium to produce a plurality of particles.

TABLE 1

| | | | | | | | Landrace |
|---|---|---|---|---|---|---|---|
| | Co- | | | | General | | Proprietary |
| Surfactant | Surfactant | Partitioning | Partitioning | Clathrate | Clarifying | Soother- | Cannabinoid |
| Solubilizer | Solubilizer | Solubilizer | Solubilizer | Solubilizer | Solubilizer | Sensates | Blends |
| Non-Ionic | Ionic | DMI | Transcutol | beta- | Regular | Flavors/ | Unique |
| | | | HP | Cyclodextrin | | Fragrances | |
| 1 to 10% | 0.01 to 10% | 0.001 to 80% | 0.001 to 70% | 0.001 to 30% | 0.01 to 80% | 0.001 to 20% | 0.001 to 30% |
| w/w | w/w | w/w | w/w | w/w | w/w | w/w | w/w |
| poloxamer | soy lecithin, | dimethyl | transcutol HP | beta- | 1,3- | natural and | Landrace Blend |
| 407/334/184, | phosphatidyl | isosorbide | | cyclodextrin | propanediol, | artificial | 100-300 |
| polysorbate | choline, | | | | propylene | flavors and | |
| 80, 60, 20. | phospholipids | | | | glycol, glycerin, | fragrances, | |
| polyoxyl castor | | | | | triacetin, | terpenes. | |
| oil 200, 40, 35, | | | | | diacetin, | | |
| 29 | | | | | triethylcitrate | | |

The self-emulsifying composition and formulations form an emulsion when placed in an aqueous medium, wherein the emulsion is stable for at least four hours. In certain embodiments, the aqueous medium is water. In certain embodiments, the aqueous medium is an intestinal fluid. In certain embodiments, the aqueous medium is a gastrointestinal fluid, or simulated intestinal or gastric fluids.

Active cannabinoid ingredients for the composition may be purchased, synthesized using well-known techniques, or extracted from a cannabis plant using well-known methods. Suitable cannabinoids and cannabinoid extracts are available from Landrace Bioscience Inc. of Chattanooga, Tenn. The relative amount of each cannabinoid in the plant extract, e.g., cannabis extract, varies according to the cannabinoid profile and levels of the particular plants and methodology of extraction. Further, the cannabis-based preparation may include a cannabis resin. The cannabis resin is selected from the group that includes: non-distilled CBD resin; non-distilled THC resin; THC distilled resin; and CBD distilled resin

In one embodiment, the active ingredient may be an extract from a cannabis plant (“cannabis extract”). Cannabis plants belong to the family Cannabaceae, preferably Cannabis sativa, Cannabis indica, or Cannabis hybrid. The cannabis extract may comprise one or more cannabinoids or other actives. In certain embodiments, the pharmaceutical composition comprises about 0.01% to about 30% by weight of a cannabinoid or a mixture of cannabinoids. In certain embodiments, the pharmaceutical composition comprises about 2% to about 20% by weight of a cannabinoid or a mixture of cannabinoids. In certain embodiments, the pharmaceutical composition comprises about 2% to about 10% by weight of a cannabinoid or a mixture of cannabinoids. In certain embodiments, the cannabinoid is selected from the group consisting of cannabidiol (CBD), tetrahydrocannabinoid (THC) or combinations thereof.

The non-ionic surfactant component of the pharmaceutical formulations can be used either alone, or in combination with an ionic co-surfactant to enhance the self-emulsifying properties of the formulation. In certain embodiments, the pharmaceutical composition comprises about 0.03% to about 40% by weight of the non-ionic surfactant solubilizer. In certain embodiments, the pharmaceutical composition comprises an amount of non-ionic surfactant of from about 8.5% to about 30% by weight. In certain embodiments, the pharmaceutical composition comprises an amount of ionic co-surfactant of from about 9% to about 22% by weight. In certain embodiments, the ionic co-surfactant is selected from the group consisting of poloxamer 184, poloxamer 334, poloxamer 407, polysorbate 20, polysorbate 60, polysorbate 80, polyoxol 29, polyoxol 35, polyoxol 40, polyoxol 200, and combinations thereof.

In certain embodiments, the pharmaceutical composition comprises about 0.1% to about 15% by weight of the ionic surfactant solubilizer. In certain embodiments, the pharmaceutical composition comprises an amount of ionic surfactant solubilizer of from about 0.5% to about 5% by weight. In certain embodiments, the ionic emulsifier is selected from the group consisting of from soy lecithin, phosphatidyl choline, phospholipids, and combinations thereof.

The partitioning solubilizer component of the pharmaceutical formulations can be used either alone, or in combination with a second partitioning solubilizer. In certain embodiments, the pharmaceutical composition comprises about 0.01% to about 80% by weight of the first partitioning solubilizer. In certain embodiments, the pharmaceutical composition comprises an amount of the first partitioning solubilizer of from about 25% to about 75% by weight. In certain embodiments, the pharmaceutical composition comprises an amount of the first partitioning solubilizer of from about 42% to about 63% by weight. In certain embodiments, the first partitioning solubilizer comprises dimethyl isosorbide (DMI).

In certain embodiments, the pharmaceutical composition comprises about 0.01% to about 70% by weight of the second partitioning solubilizer. In certain embodiments, the pharmaceutical composition comprises an amount of the second partitioning solubilizer of from about 14% to about 50% by weight. In certain embodiments, the pharmaceutical composition comprises an amount of the second partitioning solubilizer of from about 20% to about 29% by weight. In certain embodiments, the second partitioning solubilizer comprises transcutol HP.

In certain embodiments, the pharmaceutical composition comprises about 0.01% to about 50% by weight of the clathrate solubilizer. In certain embodiments, the pharmaceutical composition comprises an amount of the clathrate solubilizer of from about 5% to about 20% by weight. In certain embodiments, the clathrate solubilizer comprises beta cyclodextrin.

In certain embodiments, the pharmaceutical composition comprises about 0.01% to about 80% by weight of the general solubilizer. In certain embodiments, the pharmaceutical composition comprises an amount of the general solubilizer of from about 10% to about 60% by weight. In certain embodiments, the pharmaceutical composition comprises an amount of the general solubilizer of from about 15% to about 45% by weight. In certain embodiments, the general solubilizer is selected from 1,3-propanediol, propylene glycol, glycerin, triacetin, diacetin, triethylcitrate, and combinations thereof.

A pharmaceutical composition as described herein may also optionally comprise optional inactive ingredients selected from a group consisting of flavoring and other sensates, colors, preservatives, sweeteners, edible carriers, and combinations thereof. As used herein, the term “flavoring”' may represent a single species of flavor agent (e.g., limonene) or a mixture of flavor agent species (e.g., limonene, linalool, citral, citronellol, geranyl acetate and perillaidehyde) combined to produce a certain flavor. The flavoring may further comprise a vehicle, e.g., medium chain triglycerides (MCT), for solubilizing the flavor agent(s). A “flavor agent” is a single molecule, e.g., limonene, used alone or in combination with other flavor agent(s) to produce a certain flavor, e.g., citrus or orange, of a flavoring. A flavoring is normally supplied as a concentrate for dilution and for the purpose of imparting a flavor or taste-masking a substance.

In certain embodiments, the pharmaceutical composition comprises about 0.01% to about 30% by weight of the sensate. In certain embodiments, the pharmaceutical composition comprises an amount of the sensate of from about 0.1% to about 15% by weight. In certain embodiments, the pharmaceutical composition comprises an amount of the sensate of from about 0.1% to about 5% by weight. In certain embodiments, the sensate is selected from natural flavors, artificial flavors, fragrances, terpenes, and combinations thereof.

In certain embodiments of the pharmaceutical composition, the components comprising the formulations may be added in quantities that maintain a desired ratio of the ingredients. In at least some embodiments, the weight ratio between the partitioning solubilizer to total surfactant ratio is from about 3:1 to about 9:1. In certain embodiments, the ratio between the weight of cannabinoid to the volume of the formulation is greater than about 25:1.

In an aspect, a method of preparing a cannabis-based pharmaceutical formulation as described above may be provided. A mixture may be prepared according to the method comprising the following steps. The preparation method comprises heating a stainless-steel batching container to about 60° C. A cannabinoid distillate blend or other desired botanical ingredients is added to the batching container along with ethoxylated castor oil wax. The remaining ingredients, as described hereinabove, and other excipients are added dropwise into the batching container. Using a high shear homogenizer or mixer, the mixture is blended for up to 5 minutes. The blend is then transferred to a clean suitable aluminum container for bulk storage.

The pharmaceutical composition may be formulated for administration according to any known method. A non-limiting list of possibilities for administration routes are oral, dermal such as transdermal, intradermal or subcutaneous, and inhalations such as via vaporization in a spray, rectal and intraperitoneal. The pharmaceutical composition may be introduced by rechargeable or biodegradable polymeric devices or other devices such as, for example, patches and pumps, or formulations, which provide for the extended, slow or controlled release of the compound or agent.

The pharmaceutical composition may further be provided in a dosage form, comprising or consisting of any one of the compositions described above. The term “dosage form” denotes any form of the formulation that contains an amount of a cannabinoid or of a mixture of cannabinoids sufficient to achieve at least a partial therapeutic effect with a single administration. In certain embodiments, the dosage form is formulated as a hard shell capsule, a soft shell capsule, a tablet, a liquid, a syrup or enema or pessaries or ovule. In certain embodiments, the dosage form is granules or pellets delivered in a sachet or filled into capsule or compressed into a tablet. Each possibility represents a separate embodiment of the invention. In certain embodiments, the dosage form is formulated for mucosal delivery. The term “mucosal delivery” refers to the delivery to a mucosal surface, including nasal, pulmonary, vaginal, rectal, urethral, sublingual and buccal delivery. In certain embodiments, the dosage form is formulated as or in a candy, toffee, dragee, chocolate, cookie or lozenge. In another embodiment, the invention provides for an edible product comprising a composition of the present invention. Edible products include a lozenge, candy (including hard candies/boiled sweets, lollipop, gummy candy, candy bar, etc.), chocolates, brownie, cookie, trail bar, crackers, dissolving strip, mint, pastry, bread, etc. Further included is chewing gum, although the base gum is not consumed. The delivery vehicles act as a system dispersing or emulsifying agents for the liberated drug in a finely divided state.

In another aspect, a method of treating a disease or disorder in a subject, human or animal, who would benefit from cannabinoid is provided. The treatment method comprises the step of administering an effective amount of any one of the compositions, formulations or dosage forms comprising cannabionoids as described herein for treating or ameliorating of a cannabinoid-responsive symptom, disease, disorder or other medical condition in a human or animal. The symptoms or medical conditions may include one or more of inflammation, inflammatory diseases, including autoimmune inflammatory diseases such as MS, loss of appetite, nausea, vomiting, pain, chronic pain, muscle spasms, multiple sclerosis, glaucoma, AIDS, a neuropathic condition, cancer, acne, malnutrition, arthritis, chemotherapy induced nausea and vomiting, spinal cord injury epilepsy, psychiatric indications such as schizophrenia and borderline personality disorders (BPD) and opioid addiction withdrawal syndrome. It can be appreciated that cannabis based products can be used for treatment of other symptoms or other conditions and indications that are treated by high dose of cannabinoids. in another embodiment, the method is for increasing socialization, increasing relaxation, inducing sleep, reducing the time needed to fall asleep, or for inducing a psychotropic effect (commonly known as a “high”).The composition may be administered once, twice, three, or four times a day, or as needed.

### Examples

The following examples illustrate pharmaceutical formulations embodying the composition as described herein. However, the following examples are intended to be exemplary only and in no way limit the scope of the present disclosure. The listed ingredients can be suitably replaced with similar excipients known in the art.

Table 2 sets forth cannabinoid concentrate blend compositions A, C, and E before adding to a water or lotion base for liquid applications, a gummy or softchew base for soft solids applications, or chewing gum or candy base for solid applications. In certain embodiments, the pharmaceutical composition is liquid at room temperature. In certain embodiments, the pharmaceutical composition is semi-solid at room temperature. The term “semi-solid composition” as used herein is intended to mean a non-flowable composition that may be deformed when acted upon by a force. In certain embodiments, the pharmaceutical composition is solid at room temperature.

TABLE 2

| | Comp. A | Comp. C | Comp. E |
|---|---|---|---|
| Ingredient | (% w/w) | (% w/w) | (% w/w) |
| Landrace Cannabinoid Blend | 4.64 | 4.52 | 18.28 |
| Dimethyl Isosorbide | 52.98 | 27.64 | 51.08 |
| Transcutol HP | 26.49 | 50.25 | — |
| Poloxamer 184 | 15.89 | 11.31 | — |
| Polyoxyl 40 Ethoxylated Castor oil 40 | — | 6.28 | 30.64 |
| Natural/Artificial sweetener | qs | qs | qs |
| Natural/Artificial Flavor | qs | qs | qs |
| Select Active Pharmaceutical Ingredient(s) | qs | qs | qs |
| Natural Terpene Blend | qs | qs | qs |
| Natural Herb Extract | qs | qs | qs |
| TOTAL | 100 | 100 | 100 |
| Partitioning Solubilizer/Surfactant Solubilizer Ratio | 5.00 | 4.43 | 1.67 |

Table 3 sets forth cannabinoid concentrate blend compositions B, D, F and K before adding to a water or lotion base for liquid applications, a gummy or softchew base for soft solids applications, or chewing gum or candy base for solid applications.

TABLE 3

| | Comp. B | Comp. D | Comp. F | Comp. K |
|---|---|---|---|---|
| Ingredient | (% w/w) | (% w/w) | (% w/w) | (% w/w) |
| Landrace Cannabinoid Blend | 7.26 | 7.16 | 5.03 | 4.91 |
| Dimethyl Isosorbide | 60.48 | 81.80 | 55.87 | 70.96 |
| Transcutol HP | 20.16 | — | 27.93 | 14.19 |
| Polysorbate 80 | 4.04 | — | — | 2.84 |
| Polysorbate 20 | — | 4.91 | — | — |
| Polyoxyl Ethoxylated Castor oil 40 | 8.06 | — | — | 7.10 |
| Polyoxyl Ethoxylated Castor oil 200 | — | 6.13 | — | — |
| Soy Lecithin | — | — | 11.17 | — |
| Natural/Artificial sweetener | qs | qs | qs | qs |
| Natural/Artificial Flavor | qs | qs | qs | qs |
| Select Active Pharmaceutical Ingredient(s) | qs | qs | qs | qs |
| Natural Terpene Blend | qs | qs | qs | qs |
| Natural Herb Extract(s) | qs | qs | qs | qs |
| TOTAL | 100 | 100 | 100 | 100 |
| Partitioning Solubilizer/Surfactant Solubilizer Ratio | 6.66 | 7.41 | 7.50 | 8.57 |

Table 4 sets forth cannabinoid concentrate blend compositions M, N, O and P before adding to a water or lotion base for liquid applications, a gummy or softchew base for soft solids applications, or chewing gum or candy base for solid applications.

TABLE 4

| | | Comp. L | Comp. M | Comp. N | Comp. O | Comp. P |
|---|---|---|---|---|---|---|
| Ingredient | Trade Name | (% w/w) | (% w/w) | (% w/w) | (% w/w) | (% w/w) |
| Landrace CBD Isolate | “Laodrace CBD TN | 2.91% | 2.95% | 7.26% | 9.32% | 9.59% |
| | Domesticate Isolate” | | | | | |
| Dimethyl Isosorbide | Arlasolve DMI-LQ-(MH) | 63.11% | | 60.48% | 42.37% | |
| Super Refined Dimethyl | Super Refined | | 59.07% | | | 41.10% |
| Isosorbide | Arlasolve DMI-LQ-(MH) | | | | | |
| Transcutol High Purity | Transcutol HP | 24.27% | 25.32% | 20.16% | 28.25% | 27.39% |
| Polyoxyl 40 Ethoxylated Castor | Croduret 40-SS-(RB) | 6.47% | 5.91% | 8.06% | 11.58% | 10.96% |
| oil 40 | | | | | | |
| Polysorbate 80 | Polysorbate 80-LQ-((MH) | 3.24% | | 4.04% | | |
| Super Refined | Super Refined | | 6.75% | | 8.48% | 10.96% |
| Polysorbate 80 | Polysorbate 80-LQ-(MH) | | | | | |
| Select Active Pharmaceutical | TBD | qs | qs | qs | qs | qs |
| Ingredient(s) | | | | | | |
| Natural/Artificial sweetener | TBD | qs | qs | qs | qs | qs |
| Natural/Artificial Flavor | TBD | qs | qs | qs | qs | qs |
| Natural Terpene Blend | TBD | qs | qs | qs | qs | qs |
| Natural Herb Extract | TBD | qs | qs | qs | qs | qs |
| Natural Herb Extract(s) | TBD | qs | qs | qs | qs | qs |
| TOTAL | | 100 | 100 | 100 | 100 | 100 |
| Partitioning Solubilizer/ | | 9 | 6.66 | 6.66 | 3.52 | 3.12 |
| Surfactant Solubilizer Ratio | | | | | | |

Table 5 summarizes sleep and relaxation cannabinoid concentrate blend composition examples.

TABLE 5

| | | LBS-A | LBS-B |
|---|---|---|---|
| Ingredient | | Oral High End | Oral High End |
| Applications | Trade Name | Relaxation | Sleep & Relaxation |
| Landrace CBD Isolate | “Landrace CBD TN Domesticate Isolate” | 2.97% | 2.88% |
| Dimethyl Isosorbide | Arlasolve DMI-LQ-(MH) | 59.35% | 60.49% |
| Transcutol High Purity | Transcutol HP | 14.84% | 14.40% |
| Polyoxyl 40 Ethoxylated Castor oil 40 | Croduret 40-SS-(RB) | 11.87% | 8.64% |
| Super Refined Polysorbate 80 | Super Refined Polysorbate 80-LQ-(MH) | 5.93% | 6.72% |
| Melatonin | Melatonin | | 0.96% |
| Neotame | Neotame | 0.10% | 0.14% |
| Flavor | Relaxing Flavor | 1.98% | 2.88% |
| Flavor | Taste Bending F159 | 2.96% | 1.92% |
| Flavor | Taste Masking Q93 | | 0.97% |
| | | 100.00% | 100.00% |
| Partitioning Solubilizer/Surfactant Solubilizer Ratio | | 4.17 | 4.88 |

Table 6 presents pain relief and relaxation cannabinoid concentrate blend composition examples.

TABLE 6

| | | LBS-C | LBS-D |
|---|---|---|---|
| Ingredient | | Topical | Topical |
| Applications | Trade Name | Relaxation | Pain Relief & Relaxation |
| Landrace CBD Isolate | “Landrace CBD TN Domesticate Isolate” | 3.09% | 2.77% |
| Dimethyl Isosorbide | Arlasolve DMI-LQ-(MH) | 61.85% | 58.20% |
| Transcutol High Purity | Transcutol HP | 15.46% | 13.86% |
| Polyoxyl 40 Ethoxylated Castor oil 40 | Croduret 40-SS-(RB) | 12.37% | 8.31% |
| Super Refined Polysorbate 80 | Super Refined Polysorbate 80-LQ-(MH) | 6.19% | 6.47% |
| Menthol | Menthol | | 9.24% |
| Fragrance | Relaxing Fragrance | 0.52% | 0.46% |
| Fragrance | Masking Fragrance | 0.52% | 0.46% |
| Fragrance | Soothing Fragrance | | 0.23% |
| | | 100.00% | 100.00% |
| Partitioning Solubilizer/Surfactant Solubilizer Ratio | | 4.17 | 4.88 |

The pharmaceutical composition and formulations as described herein have many advantages, including improved dissolution, stability, and bioavailablitiy in cannabinoid formulations for the administration of cannabinoids to patients for improving patient compliance. The self-emulsifying cannabinoid nanodelivery system” (SECNDS) product format represents the most advanced delivery system for highly water-insoluble cannabidiol and other cannabinoids. When formulated in liquid form, the cannabinoid drug actives are easily delivered into various consumer-friendly forms. The present cannabinoid nanodelivery system technologies work for multiple forms of consumer products in addition to tablets and capsules. These forms range from liquids to gels to soft solids, which are all consumer-friendly product forms.

1. A self-emulsifying formulation comprising:
from about 0.001% to about 40% by weight of a cannabinoid or a cannabinoid blend;
from about 1% to about 10% by weight of a non-ionic surfactant solubilizer;
from about 0.1% to about 10% by weight of an ionic co-surfactant solubilizer;
from about 0.001% to about 80% by weight of a first partitioning solubilizer;
from about 0.001% to about 70% by weight of a second partitioning solubilizer;
from about 0.001% to about 30% by weight of a clathrate solubilizer;
from about 0.01% to about 80% by weight of a clarifying solubilizer; and
from about 0.001% to about 20% by weight of a sensate.

2. The formulation as recited in claim 1 wherein the cannabinoid or cannabinoid blend is an amount of from about 0.01% to about 30% by weight.

3. The formulation as recited in claim 1 wherein the cannabinoid or cannabinoid blend is an amount of from about 2% to about 20% by weight.

4. (canceled)

5. The formulation as recited in claim 1 wherein the non-ionic surfactant solubilizer is an amount of from about 0.03% to about 40% by weight.

6-7. (canceled)

8. The formulation as recited in claim 1 wherein the ionic surfactant solubilizer is an amount of from about 0.1% to about 15% by weight.

9. (canceled)

10. The formulation as recited in claim 1, wherein the partitioning solubilizer is an amount of from about 0.01% to about 80% by weight.

11-12. (canceled)

13. The formulation as recited in claim 1, wherein the second partitioning solubilizer is an amount of from about 0.01% to about 70% by weight.

14-15. (canceled)

16. The formulation as recited in claim 1, wherein the clathrate solubilizer is an amount of from about 0.01% to about 50% by weight.

17. (canceled)

18. The formulation as recited in claim 1, wherein the general solubilizer is an amount of from about 0.01% to about 80% by weight.

19-20. (canceled)

21. The formulation as recited in claim 1, wherein the sensate is an amount of from about 0.01% to about 30% by weight.

22-23. (canceled)

24. The formulation as recited in claim 1, wherein the ratio of partitioning solubilizer to total surfactant ratio is from about 1.5 to about 10.

25. (canceled)

26. The formulation as recited in claim 1, wherein the ratio of weight of cannabinoid to volume of the formulation is greater than about 25.

27. The formulation as recited in claim 1, wherein the non-ionic surfactant solubilizer is selected from poloxamer 184, poloxamer 334, poloxamer 407, polysorbate 20, polysorbate 60, polysorbate 80, polyoxol 29, polyoxol 35, polyoxol 40, polyoxol 200, and combinations thereof.

28. The formulation as recited in claim 1, wherein the ionic surfactant solubilizer is selected from soy lecithin, phosphatidyl choline, phospholipids, and combinations thereof.

29. The formulation as recited in claim 1, wherein the first partitioning solubilizer comprises dimethyl isosorbide.

30. The formulation as recited in claim 1, wherein the second partitioning solubilizer comprises transcutol HP.

31. The formulation as recited in claim 1, wherein the clathrate solubilizer comprises beta-cyclodextrin.

32. The formulation as recited in claim 1, wherein the clarifying solubilizer is selected from 1,3-propanediol, propylene glycol, glycerin, triacetin, diacetin, triethylcitrate, and combinations thereof.

33. The formulation as recited in claim 1, wherein the sensate is selected from natural flavors, artificial flavors, fragrances, terpenes, and combinations thereof.

34. A method of treating a subject, the method comprising the steps of providing a pharmaceutical composition according to any one or more of the preceding claims, and administering a therapeutically effective amount of the composition to the subject for treatment of cannabis-responsive indications.
